SEQUENCE LISTING (22 sequences)

SEQ ID NO: 1 - protein, 452 aa, Artificial Sequence
  Amino acid sequence identified using molecular biology techniques.
QVQLQESGPGLVKPSETLSLTCTVSGFSLSTSGMGVGWIRQPPGKGLEWIGHIWWDDDKYYNPSLKSRVTISVDTSKNQFSLKLSSVTAADTAVYYCARSYDDYLYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Amino acid sequence identified using molecular biology techniques.
DIQMTQSPSSLSASVGDRVTITCRASQSISDYLHWYQQKPGKAPKLLIKSVSQSISGVPSRFSGSGSGTDFTLTISSLQPEDFATYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 456 aa, Artificial Sequence
  Amino acid sequence identified using molecular biology techniques.
QVQLVQSGAEVKKPGSSVKVSCKASGYTFTSYGITWVRQAPGQGLEWMGENYPRSGNIYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 218 aa, Artificial Sequence
  Amino acid sequence identified using molecular biology techniques.
DIVMTQSPDSLAVSLGERATINCKASKKVTIFGSTSALHWYQQKPGQPPKLIYNLAKPESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 451 aa, Artificial Sequence
  Amino acid sequence identified using molecular biology techniques.
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Amino acid sequence identified using molecular biology techniques.
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 233 aa, Homo sapien
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 189 aa, Homo sapien
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 9 - protein, 525 aa, Homo sapien
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 10 - protein, 212 aa, Homo sapien
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - protein, 146 aa, Homo sapien [sequence omitted]

SEQ ID NO: 12 - protein, 193 aa, Homo sapien
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 13 - protein, 159 aa, Homo sapien
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 14 - protein, 171 aa, Homo sapien
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 15 - protein, 207 aa, Homo sapien
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 16 - protein, 182 aa, Homo sapien
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 17 - protein, 164 aa, Homo sapien
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 312 aa, Homo sapien
MAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALEENELTSLAEDLLGLTDPNGGLA

SEQ ID NO: 19 - protein, 107 aa, Homo sapien
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 20 - protein, 114 aa, Homo sapien
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 21 - protein, 107 aa, Homo sapien
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 22 - protein, 107 aa, Homo sapien
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

(19) United States
(12) Patent Application Publication
(10) Pub. No.: US 2015/0209319 A1
(43) Pub. Date: Jul. 30, 2015

(54) COMPOSITIONS COMPRISING AN ANTIBODY AND CAMOSTAT MESYLATE (CM)

(71) Applicant: Glaxo Group Limited, Brentford, Middlesex, MI (US)

(72) Inventors: Sean Matthew Cleveland, Stevenage (GB); Stefan Salomon, Stevenage (GB)

(21) Appl. No.: 14/422,707

(22) PCT Filed: Aug. 21, 2013
(86) PCT No.: PCT/IB2013/001818

§ 371 Date: Feb. 20, 2015

Related U.S. Application Data
(60) Provisional application No. 61/691,448, filed on Aug. 21, 2012.

(51) Int. Cl. A61K 31/245; A61K 39/395

(52) U.S. Cl. CPC A61K 31/245; A61K 39/3955

(57) ABSTRACT

The present disclosure provides a means of stabilising an antibody, in particular in protease-rich environments such as the stomach and intestine. A composition, in particular a pharmaceutical composition, comprising an antibody and camostat mesylate is provided, together with uses of said composition as a medicament and in methods of treatment. Compositions of the disclosure are particularly useful in the topical treatment of gastrointestinal conditions, such as Crohn's Disease or ulcerative colitis, or for direct activity in the gut mucosal immune system.

### BACKGROUND OF THE DISCLOSURE

The vast majority of biopharmaceuticals, particularly therapeutic antibodies and their fragments, are administered by the parenteral route, e.g. by intravenous or subcutaneous injection. These routes of administration can often be inconvenient and painful which reduces patient compliance, particularly when multiple injections per day are required. They can also be costly to health care providers, in terms of staff hours, storage and equipment.

Oral administration of biopharmaceuticals would overcome many of these drawbacks but has its own challenges. In particular, such molecules are subject to proteolytic degradation in the protease-rich environment of the stomach and intestine.

Importantly, there is a need for oral therapeutics that treat diseases of the gastrointestinal (GI) tract. In particular there is a need for lower doses of drug to be used to lower the risk of systemic toxicity.

Thus, there is a strong need to stabilise proteins in order to allow them to withstand the protease-rich environment of the gastrointestinal tract thus enabling the successful oral administration of biopharmaceuticals.

### SUMMARY OF THE DISCLOSURE

The disclosure provides a composition, optionally a pharmaceutical composition, comprising camostat mesylate and an antibody.

A composition of the disclosure for use as a medicament is provided. The use of a composition of the disclosure for the manufacture of a medicament is also provided. In particular the composition is to be administered orally.

The disclosure provides a method of treating a gastrointestinal condition comprising the step of administering, optionally orally, a composition of the disclosure to a patient in need thereof.

The disclosure further provides a method of stabilising an antibody in a protease-rich solution comprising formulating the antibody in a composition comprising camostat mesylate prior to exposing the composition to a protease-rich solution.

### BRIEF DESCRIPTION OF THE FIGURES

FIG. 1 shows the lack of stability of monoclonal antibodies (mAbs) in SIF: (a) shows a representative SDS-PAGE gel, (b) shows the half-lives of the mAbs tested.

FIG. 2 shows the ability of CM to stabilise mAbs in SIF: (a) shows a representative SDS-PAGE gel, (b) shows the half-lives of the mAbs tested, in the presence and absence of CM.

FIG. 3 shows the binding of intact mAb to its ligand in an ELISA: (a) shows the results of an anti-IL6 binding assay, (b) shows the results of an anti-IL23 binding assay. Results are expressed as a percentage of the starting mAb (Oh time-point=100%).

### DETAILED DESCRIPTION

The present disclosure provides a solution to the problems discussed above. The present disclosure provides a means of stabilising an antibody. A composition, in particular a pharmaceutical composition, comprising an antibody and camostat mesylate is provided, together with uses of said composition as a medicament and in methods of treatment. The Examples herein show that camostat mesylate (CM) can be used to stabilise monoclonal antibodies in fasted simulated intestinal fluid. The antibodies retain both structural integrity and binding capability. Accordingly, the data is supportive of the use of CM for the oral delivery of biopharmaceuticals for topical treatment of GI conditions, such as Crohns' Disease or ulcerative colitis, and for direct activity in the gut mucosal immune system.

The chemical name for camostat mesylate (CAS No: 59721-29-8) is 4-[[4-[(Aminoiminomethyl)amino]benzoyl]oxy]benzeneacetic acid 2-(dimethylamino)-2-oxoethyl ester methanesulfonate and it can be obtained, for example, from Sequoia Research Products. Camostat mesylate (CM) is an orally active serine protease inhibitor, which is licensed in Japan and Korea for the treatment of pancreatitis and post-operative reflux oesophagitis (Foipan Product information sheet; Takasugi et al., Digestion 1982, 24:36-41; Kono et al., Am 3 Surg. 2005 Sep, 190(3): 412-7). CM has a broad spectrum of inhibition, including trypsin, thrombin, kallikrein and plasmin (Tamura et al., 1977, Biochimica et Biophysica Acta 484, 417-422). The metabolism of CM within the gut is not clear, however the metabolite GBPA is itself active (Beckh et al., Res Exp Med, 1987, 187: 401-406).

The term “antibody” as used herein refers to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies. In an embodiment the antibody is a monoclonal antibody. In an embodiment the antibody is a humanised antibody. In an embodiment the antibody is a human antibody.

An anti-target antibody, e.g. an anti-TNFα antibody, refers to an antibody which binds target, e.g. TNFα. The target may be any suitable target. In an embodiment an antibody of the disclosure targets any one of the following: TNFα, IL-23, LAG-3, IL-6, IL-13, IL-18, TSLP, CD3 or a receptor of any one of the foregoing, e.g. a TNFα receptor, such as TNFRαRI or TNFRαRII, an IL-23 receptor, a LAG-3 receptor, an IL-6 receptor, an IL-13 receptor, an IL-18 receptor, a TSLP receptor, or a CD3 receptor. In an embodiment an antibody of the disclosure targets a chemokine or a chemokine receptor e.g. a glutamic acid-leucine-arginine receptor i.e. an ELR receptor such as one comprising the amino acid sequence shown in SEQ ID NO:s 12 and 19-22. In an embodiment, the target is a human target e.g. human TNFα.

Affinity is the strength of binding of one molecule, e.g. an antibody of the disclosure, to another, e.g. its target, at a single binding site. The binding affinity of an antibody to its target may be determined by equilibrium methods (e.g. enzyme-linked immunoabsorbent assay (ELISA) or radioimmunoassay (RIA)), or kinetics (e.g. BIACORE™ analysis).

In an embodiment, the equilibrium dissociation constant (KD) of the antibody-target interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M⁻¹. A skilled person will appreciate that the larger the KA numerical value, the stronger the binding.

The dissociation rate constant (kd) or “off-rate” describes the stability of the antibody-target complex, i.e. the fraction of complexes that decay per second. For example, a kd of 0.01 s⁻¹ equates to 1% of the complexes decaying per second. In an embodiment, the dissociation rate constant (kd) is 1×10⁻³ s⁻¹ or less, 1×10⁻⁴ s⁻¹ or less, 1×10⁻⁵ s⁻¹ or less, or 1×10⁻⁶ s⁻¹ or less. The kd may be between 1×10⁻⁵ s⁻¹ and 1×10⁻⁴ s⁻¹; or between 1×10⁻⁴ s⁻¹ and 1×10⁻³ s¹.

The term “neutralises” as used throughout the present specification means that the biological activity of target is reduced in the presence of an antibody as described herein in comparison to the activity of target in the absence of the antibody, in vitro or in vivo. Neutralisation may be due to one or more of blocking the target binding to its receptor, preventing target from activating its receptor, down regulating the target or its receptor, or affecting effector functionality. In an embodiment, an antibody of the disclosure neutralises its target.

“Oral administration” as used herein refers to the administration of compositions as disclosed herein by mouth. Compositions of the disclosure are typically swallowed and travel into the gastrointestinal (GI) tract where they act.

The “gastrointestinal (GI) tract” includes the upper GI tract: mouth, pharynx, oesophagus and stomach; and the lower GI tract: small intestine, duodenum, jejunum, ileum, large intestine (cecum, colon—including the ascending colon, transverse colon, descending colon and sigmoid flexure), rectum and anus; as well as the gall bladder, liver and pancreas. Compositions of the disclosure may target any one or more of the aforementioned regions of the GI tract. In an embodiment, compositions target the small intestine. In an embodiment, compositions target the large intestine.

Pharmaceutical compositions disclosed herein may be for the treatment of any one or more of the human diseases described herein. In one embodiment, the pharmaceutical composition comprises an antibody optionally in combination with one or more pharmaceutically acceptable carriers and/or excipients.

Such compositions comprise a pharmaceutically acceptable carrier as known and called for by acceptable pharmaceutical practice, see e.g. Remingtons Pharmaceutical Sciences, 16th edition (1980) Mack Publishing Co. Methods for the preparation of such pharmaceutical compositions are well known to those skilled in the art.

In an embodiment, pharmaceutical compositions of the disclosure are to be administered orally. A variety of dosage forms are contemplated, including liquids (solutions, suspensions (aqueous or oily), and emulsions), semi-solids (pastes), films and solids (tablets, lozenges, capsules, powders, crystals and granules).

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol.

Pharmaceutical compositions, in particular solid compositions such as tablets and capsules, may be enterically coated. Materials used for enteric coatings include fatty acids, waxes, shellac, plastics, and plant fibres. Suitable enteric coatings are disclosed in the EURDAGIT® Application Guidelines (11^{th} edition, 09/2009).

Effective doses and treatment regimes for administering the antibody may be dependent on factors such as the age, weight and health status of the patient and disease to be treated. Such factors are within the purview of the attending physician. Guidance in selecting appropriate doses may be found in e.g. Smith et al (1977) Antibodies in human diagnosis and therapy, Raven Press, New York.

The ratio of antibody to camostat mesylate in compositions of the disclosure may be about 1:0.1; 1:1; 1:10, 1:25, 1:50, or 1:100. In an embodiment the ratio of single variable domain to camostat mesylate in compositions of the disclosure is about 1:100. In an embodiment the ratio of single variable domain to camostat mesylate in compositions of the disclosure is about 1:10.

The pharmaceutical composition may comprise a kit of parts of the antibody together with other medicaments, optionally with instructions for use. For convenience, the kit may comprise the reagents in predetermined amounts with instructions for use.

The disclosure provides methods of treating diseases disclosed herein comprising the step of administering compositions of the disclosure to a patient in need thereof.

The present disclosure also provides the use of compositions of the disclosure as described herein in the manufacture of a medicament for the treatment of the diseases and disorders listed herein. Diseases and disorders which may be treated by compositions of the disclosure include gastrointestinal disorders.

A “gastrointestinal disorder” is a disorder affecting the GI tract and includes enteritis, proctitis, inflammatory bowel disease (IBD) including Crohn's disease, colitis including ulcerative colitis, celiac disease, Behet's syndrome and oral mucositis. In an embodiment the gastrointestinal disorder is IBD. In an embodiment the gastrointestinal disorder is Crohn's disease. In an embodiment the gastrointestinal disorder is ulcerative colitis.

Any other disease which may be treated by targeting the GI tract is encompassed within diseases to be treated by the methods of the disclosure. For example, a single variable domain of the disclosure which binds to a target within the GI tract may result in effects which go beyond the GI tract and result in the treatment of a systemic disease.

The terms “individual”, “subject” and “patient” are used herein interchangeably. The subject is typically a human. The subject may also be a mammal, such as a mouse, rat or primate (e.g. a marmoset or monkey). The subject can be a non-human animal.

Treatment can be therapeutic, prophylactic or preventative. The subject will be one who is in need thereof. Those in need of treatment may include individuals already suffering from a particular medical disease in addition to those who may develop the disease in the future. A therapeutically effective amount of the antibody described herein is an amount effective to ameliorate or reduce one or more symptoms of, or to prevent or cure, the disease.

A “protease-rich” solution is a solution comprising a protease, in particular a protease found in the GI tract, for example in a physiological amount. A protease is an enzyme that conducts proteolysis by hydrolysing one or more peptide bonds in a polypeptide chain. A physiological amount of trypsin inter-digestively in a human is 20-50 U/ml. A physiological amount of trypsin early postprandially in a human is 60-100 U/ml. A physiological amount of trypsin late postprandially in a human is 500-1500 U/ml (McConnell et al., International Journal of Pharmaceutics 364: 213-226 (2008)). In an embodiment, the trypsin amount in a protease-rich solution may be any of the aforementioned ranges. In an embodiment, the protease-rich solution comprises trypsin in an amount greater than any one of the following amounts: 20 U/ml, 30 U/ml, 40 U/ml, 50 U/ml, 60 U/ml, 70 U/ml, 80 U/ml, 90 U/ml, 100 U/ml, 200 U/ml, 300 U/ml, 400 U/ml, 500 U/ml, 600 U/ml, 700 U/ml, 800 U/ml, 900 U/ml, 1000 U/ml, 1100 U/ml, 1200 U/ml, 1300 U/ml, 1400 U/ml, or 1500 U/ml. In an embodiment, the protease-rich solution may further comprise chymotrypsin and/or pancreatin. In an embodiment, the protease-rich solution comprises trypsin, chymotrypsin and/or pancreatin. In an embodiment, the protease-rich solution is simulated intestinal fluid (SIF). SIF comprises bile, pancreatin and trypsin. SIF may also comprise sodium chloride, potassium chloride and calcium chloride. In an embodiment the SIF is as described in Example, e.g. comprising the proteases in the amounts specified in Example 1.

Within this specification the disclosure has been described, with reference to embodiments, in a way which enables a clear and concise specification to be written. It is intended and should be appreciated that embodiments may be variously combined or separated without parting from the disclosure.

### EXAMPLES

### Example 1

### Intrinsic Instability of a Panel of Monoclonal Antibodies in Simulated Intestinal Fluid (SIF)

Simulated intestinal fluid (SIF) was formulated based on a recipe used in the TNO-TIM™ gut model system, but with the volume substantially scaled down, as detailed below.

SIF Preparation

Bile solution was prepared by gently adding, with continuous stirring, 2.0 g (+/−0.02 g) of bile powder into 250 g (+/−5 g) of purified water until a clear solution was obtained.

Pancreatin solution was prepared by adding 2.1 g (+/−0.2 g) of pancreatin powder to 150 g (+/−3 g) of purified water. A stirrer was used and care was taken to minimise foaming. Once a homogenous mixture was obtained, the solution was centrifuged at 3500 rpm for 20 minutes and the supernatant was then stored on ice.

Small intestine electrolyte solution (SIES) 25% (concentrated) was produced by adding purified water to 250 g (+/−5 g) sodium chloride, 30 g (+/−0.5 g) potassium chloride, and 15 g (+/−0.3 g) calcium chloride dehydrate to make a total of 2174 g. Once the salts had dissolved the pH was adjusted to pH 7.0 (+/−0.5) with 1M sodium hydroxide.

SIES dilute was then prepared using 43.5 (+/−1 g) SIES concentrate added to purified water to a total weight of 1000 g.

Trypsin solution was prepared by dissolving 200 mg (+/−5 mg) of trypsin in 100 g (+/−2 g) of SIES dilute. This solution was then pipetted into 1.5 ml eppendorf tubes (1 ml per tube) and frozen at −20° C.

The SIF was then prepared by mixing 25 g (+/−0.3 g) of bile solution, 12.5 g (+/−0.3 g) pancreatin solution and 12.5 g (+/−0.5 g) of SIES dilute (ratio 2:1:1 bile/pancreatin/SIES dilute). 1 ml of trypsin solution was then added prior to the immediate use of the solution.

Monoclonal Antibody Preparation

Monoclonal antibodies (mAb) under investigation were concentrated to approximately 20 mg/ml using Vivaspin™ 500 50 kD MWCO columns. Columns were pre-rinsed with PBS prior to use to maximise sample recovery. Concentration was confirmed by Nanodrop™ using the IgG co-efficient option. All antibodies tested were chosen for their therapeutic potential in inflammatory bowel disease: an anti-IL-6 antibody (SEQ ID NOs: 1 and 2), an anti-IL-23 antibody (SEQ ID NOs: 3 and 4), an anti-TNFα antibody (adalimumab) (SEQ ID NOs: 5 and 6), and an anti-LAG-3 antibody.

Reaction Assembly

Incubations of mAb in SIF were carried out in a final volume of 250 μl. The volume of mAb spiked into the mixture gave a final concentration of 1 mg/ml.

A 25 μl aliquot was immediately removed and stored on dry ice (0 hour timepoint). Reaction mixtures were incubated at 37° C. with shaking (100 rpm). Subsequent 250 μl aliquots were removed at: 0.25 hours, 0.5 hours, 1 hour, 2 hours, 4 hours, 6 hours and overnight. Samples were snap frozen on dry ice and stored at −80° C. prior to analysis. The anti-IL23 mAb was incubated in SIF on a separate occasion to the other three antibodies.

SDS-PAGE Analysis

The amount of mAb remaining in the SIF at various time-points was measured by SDS-PAGE and densitometry. Briefly, sample was diluted 1/10 in a water and sample loading buffer mixture, and heated to 80° C. for 5 min. Samples were quickly chilled, then 10 μl loaded into a 4-12% Novex™ bis-tris gel along with a prepared standard (mAb in water) and a molecular weight marker. The gel was run at 150V constant in lx MOPS buffer for 75 minutes, and the protein bands visualised by staining with Instant Blue™ overnight. Densitometry of the resulting bands was performed using the Odyssey Li-Cor™ gel imaging system and the amount of mAb present calculated relative to the density of the 0 h time-point band (starting amount). An exponential curve of time vs. percentage of starting amount of mAb was prepared, and the time at which 50% of the starting amount of mAb was present was taken to be the half-life.

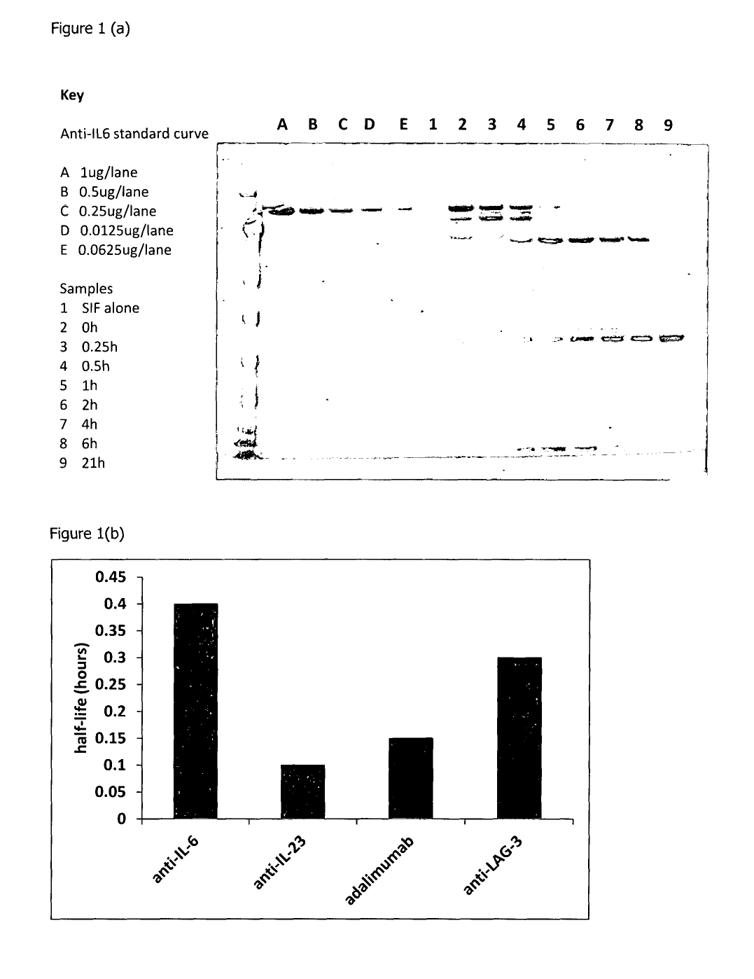

Monoclonal antibodies were found to be very susceptible to enzymatic digestion in SIF—the fully intact molecule was absent after 1 hour. The mAbs showed a distinctive pattern of bands when analysed by SDS-PAGE, suggesting that the susceptible cleavage sites were in non-variable regions of the molecule. The gel in FIG. 1 (a) shows the degradation pattern of an anti-IL6 mAb, which is representative of what was observed with the panel of mAbs.

All four mAbs tested had a half-life of less than 30 min in SIF (FIG. 1 (b)). The half-life was calculated using the density of the top band on the gel (fully intact molecule).

### Example 2

### Stabilisation of Monoclonal Antibodies Using Camostat Mesylate

The panel of mAbs studied in Example 1 was also incubated in SIF in the presence of camostat mesylate (CM, Sequoia Research Products), to determine whether inhibition of proteases would improve mAb stability. CM was added to the electrolyte solution stated above at a concentration of 350 mg/ml (CM was highly concentrated but below point of saturation) and warmed to 50° C. to dissolve. CM was added to the SIF/mAb at a final concentration of 10 mg/ml. The time-points used and analysis by SDS-PAGE were as in Example 1. As stated in Example 1, anti-IL23 was incubated in SIF with CM on a separate occasion to the other antibodies.

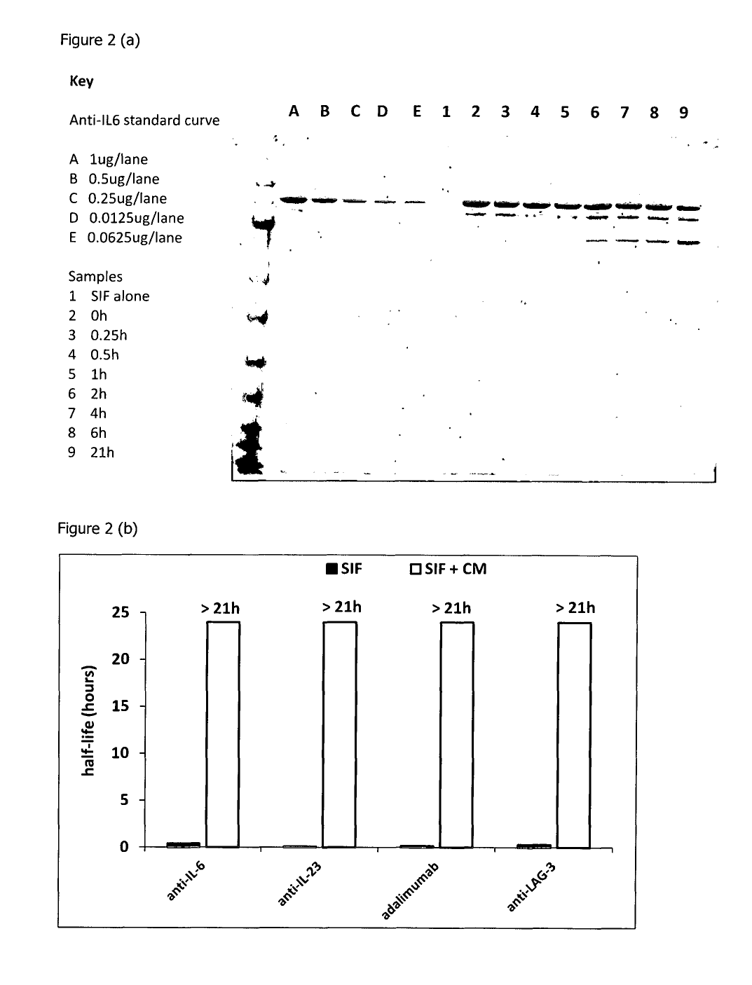

CM stabilised monoclonal antibodies, when added at 10 mg/ml. The half-life of each mAb studied was extended to over 21 hours as shown in FIG. 2 (b). The gel shown in FIG. 2 (a) shows the stabilisation of anti-IL6 mAb, which is representative of what was observed with the panel.

### Example 3

### CM-Stabilised Monoclonal Antibodies Bind to Their Target Ligand

Monoclonal antibodies were incubated in SIF in the presence and absence of CM as in Examples 1 and 2, and ability to bind to their ligand was assessed using an ELISA. In brief, Nunc Maxisorp™ plates were coated with ligand (in this Example, IL-6 and IL-23) overnight. Plates were washed and blocked with bovine serum albumin. SIF samples were diluted and added to the plate, along with a standard curve of mAb, then incubated at room temperature to allow binding. Plates were washed and a peroxidase-conjugated anti-human Fc region antibody was added to the wells. After incubation, the plate was washed and incubated with OPD substrate to obtain a colorimetric signal. The reaction was stopped with sulphuric acid and absorbance read at 490 nm.

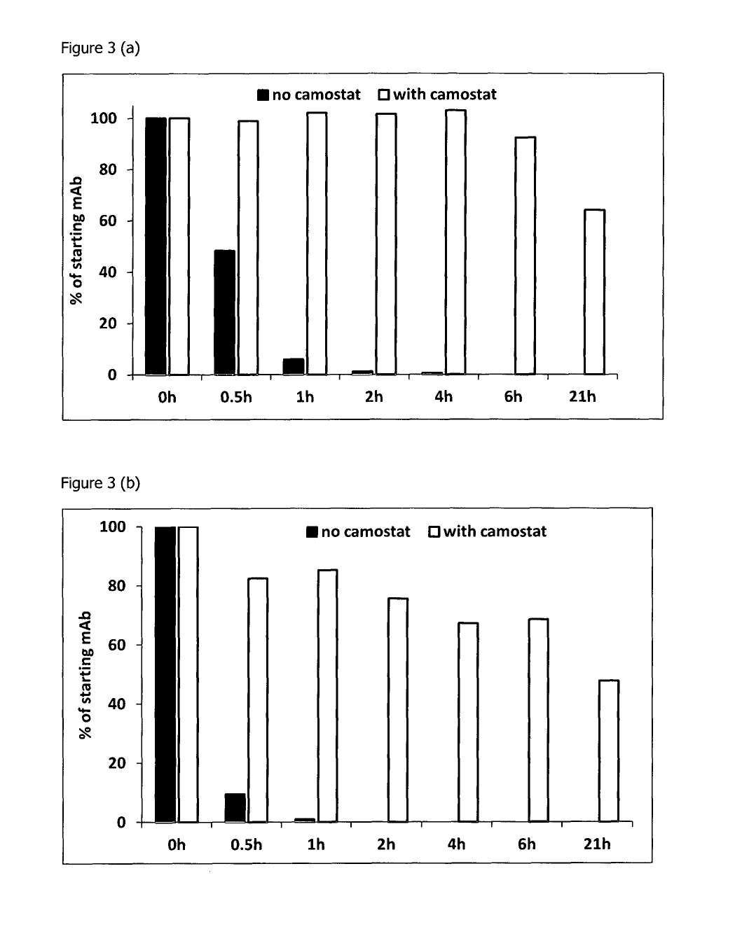

The binding of anti-IL6 (FIG. 3 (a)) and anti-IL23 (FIG. 3 (b)) to their respective ligands correlated with what was observed by SDS-PAGE. The amount of bound mAb dropped rapidly when added to SIF alone; over 90% intact bound mAb was lost by 1 hour. In contrast, addition of CM increased the amount of bound intact mAb—substantial amounts were still detectable at 21 h.

### Summary of Examples

These Examples demonstrate that camostat mesylate can stabilise a range of monoclonal antibodies in conditions that simulate what is observed in the small intestine after fasting. The stabilisation of the whole panel of mAbs under investigation suggests that CM could be used to stabilise any mAb. This would allow oral delivery of an enteric-coated (to bypass the acidic conditions of the stomach) mAb: CM formulation to the gastrointenstinal tract, e.g. small intestine, to treat conditions such as Crohn's Disease.

1. A composition comprising camostat mesylate and an antibody.

2-3. (canceled)

4. A composition as claimed in claim 1, wherein the antibody is an anti-target antibody, wherein the target is TNFα, IL-23, LAG-3, IL-6, IL-13, IL-18, TSLP, a CD3, a receptor of any one of the foregoing or an ELR receptor.

5. A composition as claimed in claim 1, wherein the antibody to camostat mesylate ratio is about 1:0.1; 1:1; 1:10, 1:25, 1:50, or 1:100.

6. A composition as claimed in claim 1, wherein the composition is enterically coated.

7-10. (canceled)

11. A method of treating a gastrointestinal condition comprising the step of administering a composition as claimed in claim 1 to a patient in need thereof.

12. A method of extending the half-life of an antibody in a protease-rich solution comprising formulating the antibody in a composition comprising camostat mesylate prior to exposing the composition to a protease-rich solution.

13. A method as claimed in claim 12, wherein the antibody to camostat mesylate ratio is about 1:0.1; 1:1; 1:10, 1:25, 1:50, or 1:100.

14. (canceled)

15. A method as claimed in claim 13, wherein the protease-rich solution is a solution comprising trypsin, chymotrypsin and/or pancreatin.
